SEQUENCE LISTING (14 sequences)

SEQ ID NO: 1 - DNA, 501 bp, Homo sapiens
tgtgacttgccagagacccactcccttgacaacagaagaactttgatgcttctagctcaaatgtccagaatctccccatcctcttgtcttatggacagacacgacttcggtttcccacaagaagaattcgacggtaaccaattccaaaaggctccagctatctctgtcttgcacgagttgatccaacaaattttcaaccttttcactaccaaggactcctccgctgcttgggacgaagatttgcttgacaagttctgtactgagctttaccaacaattgaacgacttggaagcctgtgtcatgcaagaagagagagttggagagacccctttgatgaacgctgattccattttggctgtcaagaagtacttcagaagaattaccttgtaccttactgagaagaagtactctccatgtgcttgggaggttgttagagctgaaattatgagatccttgtctttgtctactaaccttcaagaaagattgagaagaaaggagtaa

SEQ ID NO: 2 - protein, 166 aa, Homo sapiens
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 3 - DNA, 21 bp, Artificial Sequence
  primer
gactggttccaattgacaagc

SEQ ID NO: 4 - DNA, 21 bp, Artificial Sequence
  primer
gcaaatggcattctgacatcc

SEQ ID NO: 5 - protein, 11 aa, Artificial Sequence
  signal sequence
AKEEGVSLEKR

SEQ ID NO: 6 - protein, 166 aa, Artificial Sequence
  HuIFN-alpha analog IFNa-N7
CDLPETHSLDNRRTLMLLDRMNRLSPHSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 7 - DNA, 43 bp, Artificial Sequence
  primer
gaagaactttgatgcttctagctagaatgaacagattgtcccc

SEQ ID NO: 8 - DNA, 43 bp, Artificial Sequence
  primer
ggggacaatctgttcattctagctagaagcatcaaagttcttc

SEQ ID NO: 9 - DNA, 44 bp, Artificial Sequence
  primer
gatgcttctagctcaaatgaacagattgtccccacactcttgtc

SEQ ID NO: 10 - DNA, 44 bp, Artificial Sequence
  primer
gacaagagtgtggggacaatctgttcatttgagctagaagcatc

SEQ ID NO: 11 - DNA, 44 bp, Artificial Sequence
  primer
gaactttgatgcttctagctcaaatgtccagattgtccccacac

SEQ ID NO: 12 - DNA, 44 bp, Artificial Sequence
  primer
gtgtggggacaatctggacatttgagctagaagcatcaaagttc

SEQ ID NO: 13 - DNA, 44 bp, Artificial Sequence
  primer
gctcaaatgtccagaatctccccatcctcttgtcttatggacag

SEQ ID NO: 14 - DNA, 44 bp, Artificial Sequence
  primer
ctgtccataagacaagaggatggggagattctggacatttgagc

(19) United States
(12) Patent Application Publication
(10) Pub. No.: US 2003/0065148 A1
(43) Pub. Date: Apr. 3, 2003

(54) Method for expression of human interferon alpha 1 in Pichia pastoris

(76) Inventors: Lorelie H. Villarete, Alameda, CA (US); Philip T. Liu, Alameda, CA (US); Tuan V. Ta, Alameda, CA (US)

Correspondence Address: PERKINS COIE LLP, P.O. BOX 2168, MENLO PARK, CA, 94026, US

(21) Appl. No.: 10/137,127

(22) Filed: May 2, 2002

Related U.S. Application Data
(60) Provisional application No. 60/288,206, filed on May 2, 2001.

(51) Int. Cl. C07K 14/555; C12P 21/04; C12N 1/18; C12N 15/74

(52) U.S. Cl. 530/351; 435/69.51; 435/254.23; 435/320.1

(57) ABSTRACT

The invention describes a method of producing a high level of secreted, highly biologically active recombinant human Interferon Alpha 1 in a cost effective manner in Pichia pastoris.

This application claims priority to U.S. Provisional Application Serial No. 60/288,206, filed on May 2, 2001, which is incorporated herein by reference in its entirety.

### FIELD OF THE INVENTION

The present invention relates to a method of expressing human Interferon Alpha 1 in Pichia pastoris.

### REFERENCES

Akiyama, K. et al. (1993) A clinical trial of recombinant bovine interferon alpha 1 for the control of bovine respiratory disease in calves. J. Vet. Med. Sci. 55:3, 449-452.

Ausubel, F. M., et al. (1992) in Current Protocols in Molecular Biology.

Babiuk, L. A. (1987) Use of recombinant bovine alpha interferon in reducing respiratory disease induced by bovine herpes virus type 1. Antimicrob. Agents Chemother. 31:5, 752-757.

Bacila et al. eds. (1978) Biochemistry and Genetics of Yeast.

Bitter et al. (1984) Proc. Natl. Acad. Sci. 81:5330-5334.

Brake, A. J., et al. (1984) Alpha-factor-directed synthesis and secretion of mature foreign proteins in Saccharomyces cerevisiae. Proc. Natl. Acad. Sci. USA 81:4642-4646.

Breitling, R. et al. (1989) Secretory expression in Escherichia coli and Bacillus subtilis of human interferon alpha genes directed by staphylokinase signals. Mol. Gen. Genet. 217:2-3, 384-91.

Brierley, R. A. (1998) Secretion of recombinant human insulin-like growth factor 1 (IGF-1). Methods Mol. Biol. 103, 149-177.

Brocca, S., et al. (1998) Design, total synthesis, and functional overexpression of the Candida rugosa lip1 gene coding for a major industrial lipase. Protein Sci. 7, 1415-1422.

Cereghino, J. L. and Cregg, J. M. (2000) Heterologous protein expression in the methylotrophic yeast Pichia pastoris. FEMS Microbiology Reviews 24, 45-66.

Cheng, et al. (1997) The clinical study on treatment of hepatic fibrosis of hepatitis B by IFN-alpha 1 and Chinese medical preparation. Chung Kuo Chung His I Chieh Ho Tsa Chih 17:8, 453-455.

Cohen et al. (1980) Proc. Natl. Acad. Sci. USA 77:1078.

Cregg, J. M. and Madden, K. R. (1989) Use of site-specific recombination to regenerate selectable markers. Mol. Gen. Genet. 219, 320-323.

Cregg, J. M. and Russell, K. A. (1998) Transformations Methods. Mol. Biol. 103, 27-39.

Cregg, J. M., et al. (1985) Pichia pastoris as a host system for transformations. Mol. Cell. Biol. 5, 3376-3385.

Cregg, J. M., et al. (1988) Development of the methylotrophic yeast, Pichia pastoris, as a host system for the production of foreign proteins. Dev. Ind. Microbiol. 23, 33-41.

Cregg, J. M., et al. (1989) Functional characterization of the two alcohol oxidase genes from the yeast Pichia pastoris. Mol. Cell. Biol. 5, 111-1121.

Cregg, J. M., et al. (1993) Recent advances in the expression of foreign genes in Pichia pastoris. Bio/Technology 11:905-910.

Clare, J. J., et al. (1991) Production of mouse epidermal growth factor in yeast: high-level secretion using Pichia pastoris strains containing multiple gene copies. Gene 105, 205-212.

Dayhoff et al. (1978) in Atlas of Protein Sequence and Structure (Natl. Biomed. Res. Found., Washington, D.C.).

De Maeyer, E. et al. (1982) Expression of a chemically synthesized human alpha 1 interferon gene. Proc. Natl. Acad. Sci. USA 79:14, 4256-4259.

Deutscher, (1990) Methods in Enz. 182.

Elliott et al. (1983) Proc. Natl. Acad. Sci. USA 80:7080-7084

Ellis, S. B., et al. (1985) Isolation of alcohol oxidase and two other methanol regulatable genes from the yeast Pichia pastoris. Mol. Cell. Biol. 9, 1316-1323.

Henikoff et al. (1981) Nature 283:835.

Higgins, D. R., et al. (1998) Small vectors for expression based on dominant drug resistance with direct multicopy selection. Methods Mol. Biol. 103, 41-53.

Hollenberg et al. (1981) Curr. Topics Microbiol. Immunol. 96:119.

Jin, X. Y. (1992) A clinical investigation of rHuIFN alpha-1 in the treatment of herpes simplex virus keratitis. Chung Hua Yen Ko Tsa Chih 28:3, 134-137.

Julius et al. (1983) Cell 32:839-852

Kunkel (1985) Proc. Natl. Acad. Sci. USA 82:488-492.

Kunkel et al. (1987) Methods Enzymol. 154:367-382.

Liu, H., et al. (1992) An efficient screen for peroxisome-deficient mutants of Pichia pastoris. J. Bacteriol. 174, 4943-4951.

Liu, H., et al. (1995) PER3, a gene required for peroxisome biogenesis in Pichia pastoris, encodes a peroxisomal membrane protein involved in protein import. J Biol. Chem. 270, 10940-10951.

Mercereau-Puigalon et al. (1980) Gene 11:163.

Noisakran, S. and Carr, D.J.J. (2000) Plasmid DNA encoding IFN-α1 antagonizes herpes simplex virus type 1 ocular infection through CD4⁺ and CD8⁺ T Lymphocytes. Journal of Immunology 164(12):6435-43.

Noisakran, S., et al. (1999) Ectopic expression of DNA encoding IFN-α1 in the cornea protects mice from herpes simplex virus type 1-induced encephalitis. J. Immunology 162(7):4184-90.

Panthier et al. (1980) Curr. Genet. 2:109.

Pontzer C. H., et al. (1995) Measurement of interferons. Meth. Neurosci. 24:3-9.

Raemaekers, R.J.M., et al (1999) Functional phytohaemagglutinin (PHA) and Galanthus niialis agglutinin (GNA) expressed in Pichia pastoris: correct N-termninal processing and secretion of heterologous proteins expressed using the PHA-E signal peptide. Eur. J. Biochem. 65, 394-403.

Riesenberg, D. et al. (1990) High cell density fermentation of recombinant Escherichia coli expressing human interferon alpha 1. Appl. Microbiol. Biotechnol. 34:1, 77-82.

Rose and Harrison, eds. (1987) The Yeasts (2^{nd} ed.).

Sambrook, J., et al. (1989) Molecular Cloning: A Laboratory Manual. Spring Harbor, N.Y.: Cold Spring Harbor Laboratory Press.

Scopes, (1982) Protein purification: principles and practice. Springer-Verlag, New York.

Sears, I. B., et al. (1998) A versatile set of vectors for constitutive and regulated gene expression in Pichia pastoris. Yeast 14, 783-790.

Shen, S., et al. (1998) A strong nitrogen source-regulated promoter for controlled expression of foreign genes in the yeast Pichia pastoris. Gene 216, 93-102.

Shepherd, et al. (1998) Prospective randomized trial of two dose levels of interferon alpha with zidovudine for the treatment of Kaposi's sarcoma associated with human immunodeficiency virus infection: a Canadian HIV Clinical Trials Netword Study. J. Clin. Oncol. 16:5, 1736-1742.

Singh et al. (1983) Nucleic Acids Res. 11:4049-4063

Singh, A. et al. (1984) Synthesis, secretion and processing of alpha-factor-interferon fusion proteins in yeast. Nucleic Acids Res. 12:23, 8927-8938.

Smith et al. (1985) Science 229:1219-1229.

Skinner et al., eds. (1980) Biology and Activities of Yeast (Soc. App. Bacteriol. Symp. Series No. 9).

Strathern et al, eds. (1981) The Molecular Biology of the Yeast Saccharomyces.

Thill, G. P., et al. (1990) Positive and negative effects of multicopy integrated expression vectors on protein expression in Pichia pastoris. In: Proceedings of the Sixth International Symposium on the Genetics of Microorganisms (Heslot, H., et al., Eds.), Vol. 2, pp. 477-490. Societe Francaise de Microbiologie, Paris.

Tschopp, J. F., et al. (1987). Expression of the LacZ gene from two methanol-regulated promoters in Pichia pastoris. Nucleic Acids Res. 15, 3859-3876.

Van Heeke, G., et al. (1996) High yield expression and secretion of the ovine pregnancy recognition hormone interferon-τ by Pichia pastoris. J. Interferon and Cytokine Res. 16:119-126.

Walker and Gaastra, eds. (1983) Techniques in Molecular Biology (MacMillan Publishing Company, New York).

Walter, M. R., et al. (1998) Review of recent developments in the molecular characterization of recombinant alpha Interferons on the 40th anniversary of the discovery of interferon. Cancer Biotherapy and Radiophamaceuticals 13:3, 143-154.

Waterham, H. R., et al. (1996). The Pichia pastoris PER6 gene product is a peroxisomal integral membrane protein essential for peroxisome biogenesis and has sequence similarity to the Zellweger syndrome protein PAF-1. Mol. Cell. Biol. 16, 2527-2536.

Waterham, H. R., et al. (1997) Isolation of the Pichia pastoris glyceraldehyde-3-phosphate dehydrogenase gene and regulation and use of its promoter. Gene 186, 37-44.

White, C. E., et al. (1995) Large-scale expression, purification and characterization of small fragments of thrombomodulin: the roles of the sixth domain and of methionine 388. Protein Eng. 8, 1177-1187.

### BACKGROUND OF THE INVENTION

Interferons (IFNs) are a family of structurally and functionally related proteins that exhibit pleiotropic effects on the growth and function of a variety of cell types. Since their discovery as antiviral agents in 1957, IFNs have been shown to exhibit various potent immunomodulatory effects, including regulation of natural killer cell activity and modulation of major histocompatibility antigen expression, as well as antiproliferative activity against malignant cells (Walter, et al., 1998).

The major classes of IFN are IFN-α-β, -τ and -ω), which are also designated type I (acid-stable), and IFN-γ (designated as type II, acid-labile). Table 1, below, summarizes the aspects of the major classes of IFNs.

TABLE 1
Overview of the Interferons

| Aspects | Type I | Type I | Type I | Type II |
|---|---|---|---|---|
| Types | α & ω | β | τ | γ |
| Produced by: | leukocyte | fibroblast | trophoblast | lymphocyte |
| Antiviral | + | + | + | + |
| Antiproliferative | + | + | + | + |
| Pregnancy Signaling | − | − | + | − |

The IFN-α family of proteins is now known to consist of at least 14 genes, including one pseudogene and two genes that encode for the same protein. Thus, there are 12 separate IFN-α proteins produced from the 14 genes. The various IFN-α subtypes share approximately 80% identity at the amino acid sequence level.

Interferon alpha-1 (IFNAα1), also known as interferon alpha-D (IFNαD), is a type I interferon of wide research and clinical interest. Recent reports have demonstrated the efficacy of recombinant IFNαD in the treatment of various viral diseases in humans as well as in animals (Noisakran and Carr, 2000; Noisakran, et al., 1999). Due to the clinical and research interest in human IFNαAD, different expression systems have been developed and employed.

Expression of recombinant human IFNαAD (rHuIFNαD) in 1982 was described for a Methylophilus methylotrophus system and an E. coli system which both utilized the lac promoter (De Maeyer, 1982). In 1984, Genentech scientists reported a Saccharomyces cerevisiae system in which the IFNαD gene fused with the α-factor prepro signal sequence yielded a secreted IFNαD protein that had relatively low biological activity (Singh, 1984). Several years later, in 1989, secretory expression of human interferon genes in E. coli and Bacillus subtilis (B. subtilis), using the staphylokinase heterologous expression-secretion signal, was developed (Breitling, 1989). In these studies, only the B. subtilis system, and not the E. coil system, was able to secrete rHuIFNαD into the culture medium. A significant improvement on the intracellular expression of rHuIFNαD in E. coli was reported in 1990 by the use of a defined medium in a fed batch mode during fermentation that allowed for a more efficient expression of the protein at reduced specific growth rates in E. coli. However, for over eleven years there has been no significant improvement in the production of human IFNαD.

Thus, there exists a long felt but unsolved need for the production of human IFNαD with a high specific activity. Because of the shortcomings associated with current recombinant human IFNαD production methods, the inventors have set out to identify a new expression method that will yield a high level of secreted, highly biologically active recombinant rHuIFNαD in a cost effective manner.

### SUMMARY OF THE INVENTION

In one aspect, the present invention provides a method for producing human IFNαD in P. pastoris cells. The method includes transforming Pichia pastoris with a vector containing a nucleotide sequence that includes in the 5′ to 3′ direction and operably linked, a P. pastoris-recognized transcription and translation initiation region, a signal peptide sequence for a P. pastoris secreted protein, a peptide coding sequence for said human IFNαD, or variants thereof, and a P pastoris-recognized transcription and translation termination region. The transformed cells are cultured and secreted IFNαD protein is obtained from the extracellular culture medium at a specific activity of at least 1.7×10⁸ U/mg protein. The IFNαD protein is then isolated from the extracellular culture medium.

In one embodiment, the transcription and translation initiation region is the Pichia pastoris AOX1 promoter. In another embodiment, the transcription and translation initiation region is the promoter selected from the group consisting of Pichia pastoris GAP, FLD1, PEX8, and YPT1 promoters. In yet another embodiment, the signal peptide sequence is a signal peptide sequence for a Saccharomyces cerevisiae α-factor. In still another embodiment, the variant has an amino acid sequence that has at least about 95% sequence identity to the amino acid sequence of human-IFNαD. Preferably, the variant has an amino acid sequence that has at least about 98% sequence identity to the amino acid sequence of human-IFNαD. More preferably, the variant has an amino acid sequence that has at least about 99% sequence identity to the amino acid sequence of human-IFNαD. In yet, still another embodiment the transcription and translation termination region is the AOX1 termination region.

In another aspect, the invention provides an isolated human IFNαD protein. The protein is prepared by transforming Pichia pastoris with a vector that includes a nucleotide sequence that comprises in the 5′ to 3′ direction and operably linked, a P. pastoris-recognized transcription and translation initiation region, a signal peptide sequence for a P. pastoris secreted protein, a peptide coding sequence for said human IFNαD, or variants thereof, and a P pastoris-recognized transcription and translation termination region. The P. pastoris cells are cultured and secreted IFNαD protein is isolated from the extracellular culture medium at a specific activity of at least 1.7×¹⁰⁸ U/mg protein.

In one embodiment, the transcription and translation initiation region is the Pichia pastoris AOX1 promoter. In another embodiment, the transcription and translation initiation region is the promoter selected from the group consisting of Pichia pastoris GAP, FLD1, PEX8, and YPT1 promoters. In yet another embodiment, the signal peptide sequence is a signal peptide sequence for a Saccharomyces cerevisiae α-factor. In still another embodiment, the variant has an amino acid sequence that has at least about 95% sequence identity to the amino acid sequence of human-IFNαD. Preferably, the variant has an amino acid sequence that has at least about 98% sequence identity to the amino acid sequence of human-IFNαD. More preferably, the variant has an amino acid sequence that has at least about 99% sequence identity to the amino acid sequence of human-IFNαD. In yet, still another embodiment, the transcription and translation termination region is the AOX1 termination region.

These and other objects and features of the invention will be more fully appreciated when the following detailed description of the invention is read in conjunction with the accompanying drawings.

### BRIEF DESCRIPTION OF THE DRAWINGS

FIG. 1 is a map showing the features and relevant restriction sites of plasmid pPEPhIFNαD.

FIG. 2 is the complete nucleotide and amino acid sequence of human IFNαD.

FIG. 3 is a polyacrylamide gel of PCR products from recombinant Pichia colonies.

FIGS. 4A and 4B show a polyacrylamide gel and Western Blot analysis, respectively, of recombinant human IFNαD protein expressed in P. pastoris.

FIG. 5 is the N-terminal sequence of the rHuIFNαD bands shown in FIG. 4B.

FIG. 6 is an IEF gel showing purification fraction number 3.

### BRIEF DESCRIPTION OF THE SEQUENCES

SEQ ID NO: 1 is the nucleotide sequence encoding human IFNαD.

SEQ ID NO:2 is the amino acid sequence of human IFNαD.

SEQ ID NO:3 is the nucleotide sequence of the 5′AOX1 primer.

SEQ ID NO:4 is the nucleotide sequence of the 3′AOX1 primer.

SEQ ID NO:5 is the amino acid sequence of the S. cerevisiae α-factor signal sequence.

SEQ ID NO:6 is the amino acid sequence of IFNα-N7.

SEQ ID NO:7 is the nucleotide sequence of primer PM-Gd1.

SEQ ID NO:8 is the nucleotide sequence of primer PM-Gd2.

SEQ ID NO:9 is the nucleotide sequence of primer NLVg(DR).MUT1.

SEQ ID NO: 10 is the nucleotide sequence of primer NLVg(DR).MUT2.

SEQ ID NO: 11 is the nucleotide sequence of primer NLVgDRN.MUT1.

SEQ ID NO: 12 is the nucleotide sequence of primer NLVgDRN.NWT2.

SEQ ID NO: 13 is the nucleotide sequence of primer NLVgNLH.MUT1.

SEQ ID NO: 14 is the nucleotide sequence of primer NLVgNLH.MUT2.

### DETAILED DESCRIPTION OF THE INVENTION

I. Definitions

Unless otherwise indicated, all technical and scientific terms used herein have the same meaning as they would to one skilled in the art of the present invention. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

All publications and patents cited herein are expressly incorporated herein by reference for the purpose of describing and disclosing compositions and methodologies which might be used in connection with the invention.

By “yeast” is intended ascosporogenous yeasts (Endomycetales), basidiosporogenous yeasts, and yeast belonging to the Fungi Imperfecti (Blastomycetes). The ascosporogenous yeasts are divided into two families, Spermophthoraceae and Saccharomycetaceae. The later is comprised of four subfamilies, Schizosaccharomycoideae (e.g., genus Schizosaccharomyces), Nadsonioideae, Lipomycoideae, and Saccharomycoideac (e.g., genera Pichia, Kllyveromyces, and Saccharomyces). The basidiosporogenous yeasts include the genera Leucosporidium, Rhodosporidium, Sporidiobolus, Filobasidium, and Filobasidiella. Yeast belonging to the Fungi Imperfecti are divided into two families, Sporobolomycetacea (e.g., genera Sporoholomyces, Bullera) and Cryptococcaceae (e.g., genus Candida). Of particular interest to the present invention are species within the genera Pichia, Kluyveromyces, Saccharomyces, Schizosaccharomyces, and Candida. Of particular interest is the Pichia species P. pastoris. Since the classification of yeast may change in the future, for the purposes of this invention, yeast shall be defined as described in Skinner et al. In addition to the foregoing, those of ordinary skill in the art are presumably familiar with the biology of yeast and the manipulation of yeast genetics. See, for example, Bacila et al.; Rose and Harrison; Strathern et al.; herein incorporated by reference.

The nucleotide sequences of the present invention are useful for producing biologically active mature heterologous proteins of interest in a yeast host cell when operably linked to a yeast promoter. In this manner, the nucleotide sequences encoding the hybrid precursor polypeptides of the invention are provided in expression cassettes for introduction into a yeast host cell. These expression cassettes will comprise a transcriptional initiation region linked to the nucleotide sequence encoding the hybrid precursor polypeptide. Such an expression cassette is provided with a plurality of restriction sites for insertion of the nucleotide sequence to be under the transcriptional regulation of the regulatory regions. The expression cassette may additionally contain selectable marker genes.

The term “vector” refers to a nucleotide sequence that can assimilate new nucleic acids, and propagate those new sequences in an appropriate host. Vectors include, but are not limited to recombinant plasmids and viruses. The vector comprising the nucleic acid of the invention can be in a carrier, for example, a plasmid complexed to a protein, a plasmid complexed with lipid-based nucleic acid transduction systems, or other non-viral carrier systems.

A cloning or expression vector may comprise additional elements, for example, the expression vector may have two replication systems, thus allowing it to be maintained in two organisms, for example in human or insect cells for expression and in a prokaryotic host for cloning and amplification.

Both cloning and expression vectors contain a nucleic acid sequence that enables the vector to replicate in one or more selected host cells. Further, for integrating expression vectors, the expression vector contains at least one sequence homologous to the host cell genome, and preferably two homologous sequences that flank the expression construct. The integrating vector may be directed to a specific locus in the host cell by selecting the appropriate homologous sequence for inclusion in the vector. Constructs for integrating vectors are well known in the art.

Cloning and expression vectors will typically contain a selectable marker. Typical selectable marker genes encode proteins that (a) confer resistance to antibiotics or other toxins, for example, ampicillin, neomycin, methotrexate, zeocin or tetracycline, (b) complement auxotrophic deficiencies, or (c) supply critical nutrients not available from complex media, for example, the gene encoding D-alanine racemase for Bacilli.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The nucleic acid coding sequence must be “operably linked” by placing it in a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” DNA sequences are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

Promoter sequences encode either constitutive or inducible promoters. The promoters may be either naturally occurring, engineered or hybrid promoters.

As used herein, the term “human IFN-αD expression” refers to transcription and translation of the human IFN-αD gene, the products of which include precursor RNA, mRNA, polypeptide, post-translation processed polypeptide, and derivatives thereof. By way of example, assays for human IFN-αD expression include standard cytopathic protection assays, Western and Northern blot analysis and reverse transcriptase polymerase chain reaction (RT-PCR) for human IFN-αD mRNA.

As used herein, the terms “biological activity of human IFN-αD” and “biologically active human IFN-αD” refer to any biological activity associated with IFN-αD, or any fragment, derivative, or analog of IFN-αD, such as enzymatic activity, and specifically including antiviral activity which can be measured using standard cytopathic protection assays.

As used herein, the term “modified form of”, relative to proteins associated with IFN-αD, means a derivative or variant form of the native protein. That is, a “modified form of” a protein has a derivative polypeptide sequence containing at least one amino acid substitution, deletion or insertion, with amino acid substitutions being particularly preferred. The amino acid substitution, insertion or deletion may occur at any residue within the polypeptide sequence, which interferes with the biological activity of the protein. The corresponding nucleic acid sequence which encodes the variant or derivative protein is considered to be a “mutated” or “modified” form of the gene or coding sequence therefor, and is included within the scope of the invention.

By “variant” is intended a polypeptide derived from the native polypeptide by deletion (so-called truncation) or addition of one or more amino acids to the N-terminal and/or C-terminal end of the native protein; deletion or addition of one or more amino acids at one or more sites in the native polypeptide; or substitution of one or more amino acids at one or more sites in the native polypeptide. Such variants may result from, for example, genetic polymorphism or from human manipulation. Methods for such manipulations are generally known in the art.

For example, amino acid sequence variants of the polypeptide can be prepared by mutations in the cloned DNA sequence encoding the native IFNαD. Methods for mutagenesis and nucleotide sequence alterations are well known in the art. See, for example, Walker and Gaastra, eds. (1983); Kunkel (1985); Kunkel et al. (1987);and Sambrook et al. (1989). Guidance as to appropriate amino acid substitutions that do not affect biological activity of the protein of interest may be found in the model of Dayhoff et al. (1978), herein incorporated by reference. Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

In constructing variants of IFNαD, modifications will be made such that variants continue to possess the desired activity. Obviously, any mutations made in the DNA encoding the variant protein must not place the sequence out of reading frame and preferably will not create complementary regions that could produce secondary mRNA structure.

Thus proteins of the invention include the naturally occurring forms of IFNαD as well as variants thereof. These variants will be substantially homologous and functionally equivalent to the native protein. A variant of a native protein is “substantially homologous” to the native protein when at least about 80%, more preferably at least about 90%, even more preferably at least about 95%, still more preferably at least about 98%, and most preferably at least about 99% of its amino acid sequence is identical to the amino acid sequence of the native protein. A variant may differ by as few as 1, 2, 3, or 4 amino acids. By “functionally equivalent” is intended that the sequence of the variant defines a chain that produces a protein having substantially the same biological activity as the native IFNαD. Such functionally equivalent variants that comprise substantial sequence variations are also encompassed by the invention. Thus, a functionally equivalent variant of the native IFNαD protein will have a sufficient biological activity to be therapeutically useful. By “therapeutically useful” is intended effective in achieving a therapeutic goal, as, for example, protection against herpes simplex virus type 1-induced encephalitis.

Methods are available in the art for determining functional equivalence. Biological activity can be measured using assays specifically designed for measuring activity of the native IFNαD protein, including assays described in the present invention and in U.S. Pat. No. 6,204,022, which is expressly incorporated by reference in its entirety herein. Additionally, antibodies raised against the biologically active native protein can be tested for their ability to bind to the functionally equivalent variant, where effective binding is indicative of a protein having a conformation similar to that of the native protein.

From the foregoing, it can be seen how various objects and features of the invention are met.

II. Method of the Invention

The invention includes, in one aspect, a method of producing human IFNαD which has a very high specific activity. Among the myriad of ever-expanding protein expression systems available, it has been discovered that when a P. pastoris expression host is transformed with a vector containing the proper nucleic acid sequences, production of large amounts of relatively pure and functionally active human IFNαD is possible. Considered below are the steps in practicing the invention.

A. P. pastoris Host Cells

The host chosen for expression of human IFNαD will preferably be a yeast. The yeast used in the method of the present invention are species within the genera Pichia. Of particular interest is the Pichia species P. pastoris. One exemplary P. pastoris strain, X-33, is described in Example 1.

P. pastoris is capable of metabolizing methanol as its sole carbon source by inducing the production of alcohol oxidase (Cregg, 1993). Most P. pastoris expression strains have one or more auxotrophic mutations which allow for selection of expression vectors containing the appropriate selectable marker gene upon transformation. Prior to transformation, these strains grow on complex media but require supplementation with the appropriate nutrient(s) for growth on minimal media. P. pastoris strains of the present invention include those strains that grow on methanol at the wild-type rate (Mut⁺), and also those which vary with regard to their ability to utilize methanol because of deletions in one or both AOX genes. Also contemplated are protease-deficient strains that can be effective in reducing degradation of foreign proteins (Brierley, 1998; and White et al., 1995).

The selection of suitable yeast and other microorganism hosts for the practice of the present invention is within the skill of the art. When selecting yeast hosts for expression, suitable hosts may include those shown to have, inter alia, good secretion capacity, low proteolytic activity, and overall vigor. Yeast and other microorganisms are generally available from a variety of sources, including the Yeast Genetic Stock Center, Department of Biophysics and Medical Physics, University of California (Berkeley, Calif.); the American Type Culture Collection (Manassas, Va.); Northern Regional Research Laboratories (Peoria, Ill.); and vendors such as Invitrogen (San Diego, Calif.).

B. Yeast Expression Vector

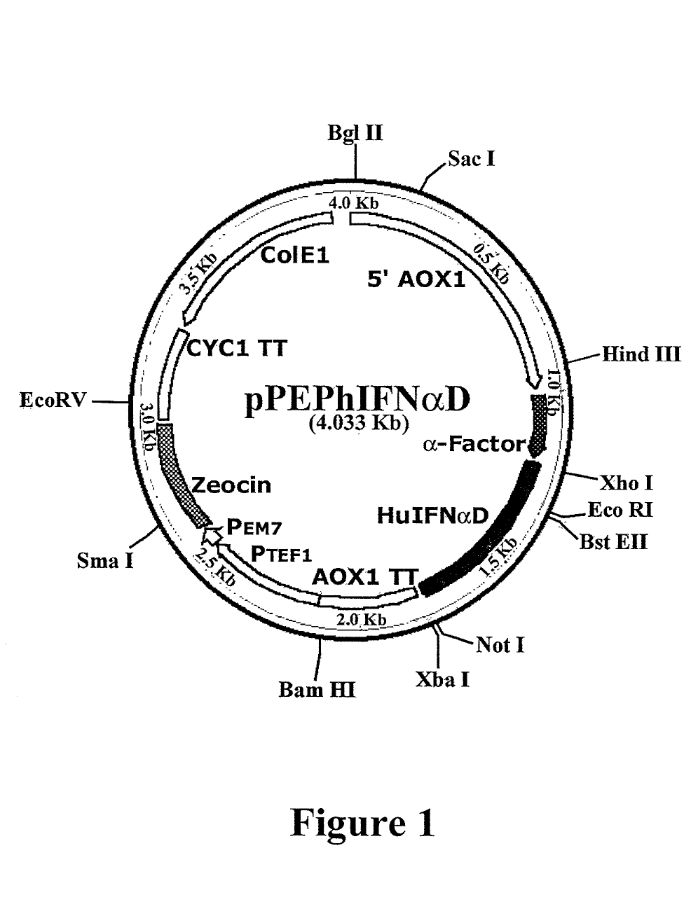

Expression vectors for use in the present invention comprise a chimeric gene (or expression cassette), designed for operation in yeast, with companion sequences upstream and downstream from the expression cassette. The companion sequences will be of plasmid or viral origin and provide necessary characteristics to the vector to permit the vectors to move DNA from bacteria to the desired yeast host. Suitable transformation vectors are described below. Suitable components of the expression plasmid, including a trancription and translation initiator, a signal sequence, a coding sequence for IFNαD, and suitable transcription and translation terminators are also discussed below. One exemplary plasmid is the pPEPhIFNαD plasmid illustrated in FIG. 1.

i. Transcription & translation initiators

The nucleotide sequences of the present invention are useful for producing biologically active mature heterologous proteins of interest in a yeast host cell when operably linked to a yeast promoter. In this manner, the nucleotide sequences encoding the hybrid precursor polypeptides of the invention are provided in expression cassettes for introduction into a yeast host cell. These expression cassettes will comprise a transcriptional initiation region linked to the nucleotide sequence encoding the hybrid precursor polypeptide. Such an expression cassette is provided with a plurality of restriction sites for insertion of the nucleotide sequence to be under the transcriptional regulation of the regulatory regions.

The transcription initiation region, the yeast promoter, provides a binding site for RNA polymerase to initiate downstream (3′) translation of the coding sequence. The promoter may be a constitutive or inducible promoter, and may be native or analogous or foreign or heterologous to the specific yeast host. Additionally, the promoter may be the natural sequence or alternatively a synthetic sequence. By foreign is intended that the transcription initiation region is not found in the native yeast of interest into which the transcription initiation region is introduced.

Suitable native yeast promoters include, but are not limited to the wild-type alpha -factor promoter, which is described in detail below, as well as other yeast promoters. Preferably the promoter is selected from the list including the P. pastoris glyceraldehyde 3-phosphate dehydrogenase (GAP), formaldehyde dehydrogenase (FLD1), peroxisomal matrix protein (PEX8), and the GTPase encoding YPT1 promoter. More preferably the promoter is the alcohol oxidase AOX1 promoter.

a. The AOX1 promoter

Although Pichia codes for two alcohol oxidase genes, AOX1 and AOX2, the AOX1 gene is responsible for the vast majority of alcohol oxidase activity in the yeast cell (Tschopp, et al., 1987; Ellis, et al, 1985; and Cregg, et al., 1989). Expression of the AOX1 gene is tightly controlled at the level of transcription by the AOX1 promoter. In methanol-grown cells, ˜5% of poly(A)⁺ RNA is from AOX1; however, in cells grown on most other carbon sources, AOX1 message is undetectable (Cregg, et al., 1988).

The regulation of the AOX1 gene appears to involve two mechanisms: a repression/derepression mechanism plus an induction mechanism, similar to the regulation of the S. cerevisiae GAL1 gene. Unlike GAL1 regulation, the absence of a repressing carbon soure, such as glucose in the medium, does not result in substantial transcription of AOX1. The presence of methanol is essential to induce high levels of transcription (Tschopp, et al., 1987).

The exemplary plasmids described in Example 2, and illustrated in FIG. 1, utilize the AOX1 promoter for protein production.

b. The GAP Promoter

Both northern and reporter activation results indicate that the P. pastoris GAP gene promoter provides strong constitutive expression on glucose at a level comparable to that seen with the AOX1 promoter (Waterham, et al., 1997). GAP promoter activity levels in glycerol- and methanol-grown cells are approximately two-thirds and one-third of the level observed for glucose, respectively. The advantage of using the GAP promoter is that methanol is not required for induction, nor is it necessary to shift cultures from one carbon source to another, making strain growth more staightforward. However, since the GAP promoter is constitutively expressed, may not be a good choice for the production of proteins that are toxic to yeast.

c. The FLD1 Promoter

The FLD1 gene encodes a glutathione-dependent formaldehyde dehydrogenase, a key enzyme required for the metabolism of certain methylated amines as nitrogen sources and methanol as a carbon source (Shen, et al., 1998). The FLD1 promoter can be induced with either methanol as a sole carbon source (and ammonium sulfate as a nitrogen source) or methylamine as a sole nitrogen source (and glucose as a carbon source). After induction with either methanol or methylamine, P_{FLD1} is able to express levels of a β-lactamase reporter gene similar to those obtained with methanol induction from the AOX1 promoter. The FLD1 promoter offers the flexibility to induce high levels of expression using either methanol or methylamine, an inexpensive nontoxic nitrogen source.

d. The PEX8 and YPT1 Promoter

For some P. pastoris strains, the AOX1, GAP, and FLD1 promoters may be too strong, expressing genes at too high a level. There is evidence that, for certain foreign genes, the high level of expression from P_{AOX1} may overwhelm the post-translational machinery of the cell, causing a significant proportion of foreign protein to be misfolded, unprocessed, or mislocalized (Thill, et al., 1990; Brierley, 1998). For these and other applications, moderately expressing promoters are desirable. Toward this end, the P. pastoris PEX8 and YPT1 promoters may be of use. The PEX8 gene encodes a peroxisomal matrix protein that is essential for peroxisome biogenesis (Liu, et al., 1995). It is expressed at a low but significant level on glucose and is induced modestly when cells are shifted to methanol. The YPT1 gene encodes a GTPase involved in secretion, and its promoter provides a low but constitutive level of expression in media containing either glucose, methanol, or mannitol as carbon sources (Sears, et al., 1998).

e. Alternative Promoters

Synthetic hybrid promoters consisting of the upstream activator sequence of one yeast promoter, which allows for inducible expression, and the transcription activation region of another yeast promoter also serve as functional promoters in a yeast host.

Yeast-recognized promoters also include naturally occurring non-yeast promoters that bind yeast RNA polymerase and initiate translation of the coding sequence. Such promoters are available in the art. See, for example, Cohen et al. (1980); Mercereau-Puigalon et al. (1980); Panthier et al. (1980); Henikoff et al. (1981); and Hollenberg et al. (1981), each of which is herein incorporated by reference.

ii. Signal sequences and leader sequences

In addition to encoding the protein of interest, the expression cassette's chimeric gene encodes a signal peptide that allows processing and translocation of the protein, as appropriate.

For purposes of the present invention, the signal peptide is a presequence that is an N-terminal sequence for the precursor polypeptide of the mature form of a yeast secreted protein. When the nucleotide sequence encoding the hybrid precursor polypeptide is expressed in a transformed yeast host cell, the signal peptide sequence functions to direct the hybrid precursor polypeptide comprising the mature heterologous protein of interest into the endoplasmic reticulum (ER). Movement into the lumen of the ER represents the initial step into the secretory pathway of the yeast host cell. The signal peptide of the invention can be heterologous or native to the yeast host cell. The signal peptide sequence of the invention may be a known naturally occurring signal sequence or any variant thereof as described above that does not adversely affect the function of the signal peptide.

During entry into the ER, the signal peptide is cleaved off the precursor polypeptide at a processing site. The processing site can comprise any peptide sequence that is recognized in vivo by a yeast proteolytic enzyme. This processing site may be the naturally occurring processing site for the signal peptide. More preferably, the naturally occurring processing site will be modified, or the processing site will be synthetically derived, so as to be a preferred processing site. By “preferred processing site” is intended a processing site that is cleaved in vivo by a yeast proteolytic enzyme more efficiently than is the naturally occurring site. Examples of preferred processing sites include, but are not limited to, dibasic peptides, particularly any combination of the two basic residues Lys and Arg, that is Lys-Lys, Lys-Arg, Arg-Lys, or Arg-Arg, most preferably Lys-Arg. These sites are cleaved by the endopeptidase encoded by the KEX2 gene of P. pastoris (Julius et al (1983). In the event that the KEX2 endopeptidase would cleave a site within the peptide sequence for the mature heterologous protein of interest, other preferred processing sites could be utilized such that the peptide sequence of interest remains intact (see, for example, Sambrook et al. (1989)).

A functional signal peptide sequence is essential to bring about extracellular secretion of a heterologous protein from a yeast cell. Additionally, the hybrid precursor polypeptide may comprise a secretion leader peptide sequence of a yeast secreted protein to further facilitate this secretion process. When present, the leader peptide sequence is generally positioned immediately 3′ to the signal peptide sequence processing site. By “secretion leader peptide sequence” is intended a peptide that directs movement of a precursor polypeptide, which for the purposes of this invention is the hybrid precursor polypeptide comprising the mature heterologous protein to be secreted, from the ER to the Golgi apparatus and from there to a secretory vesicle for secretion across the cell membrane into the cell wall area and/or the growth medium. The leader peptide sequence may be native or heterologous to the yeast host cell.

The leader peptide sequence of the present invention may be a naturally occurring sequence for the same yeast secreted protein that served as the source of the signal peptide sequence, a naturally occurring sequence for a different yeast secreted protein, or a synthetic sequence, or any variants thereof that do not adversely affect the function of the leader peptide.

a. S. cerevisiae α-factor prepro peptide

For purposes of the invention, the leader peptide sequence when present is preferably derived from the same yeast secreted protein that served as the source of the signal peptide sequence, more preferably an alpha -factor protein. A number of genes encoding precursor alpha -factor proteins have been cloned and their combined signal-leader peptide sequences identified. See, for example, Singh et al. (1983). Alpha-factor signal-leader peptide sequences have been used to express heterologous proteins in yeast. See, for example, Elliott et al. (1983); Bitter et al. (1984); and Smith et al. (1985).

Alpha-factor, an oligopeptide mating pheromone approximately 11 residues in length, is produced from a larger precursor polypeptide of between about 100 and 200 residues in length, more typically about 120-160 residues. This precursor (pre) polypeptide comprises the signal sequence, which is about 19-23 (more typically 20-22 residues), the leader sequence (pro), which is about 60-66 residues, and typically 2-6 tandem repeats of the mature pheromone sequence. Although the signal peptide sequence and full-length alpha -factor leader peptide sequence can be used, the invention also contemplates a truncated alpha-factor leader peptide sequence which can be used with the signal peptide when both elements are present in the hybrid precursor molecule.

The processing of this signal sequence involves three steps. The first is the removal of the pre signal by signal peptidase in the endoplasmic reticulum. Second, Kex2 endopeptidase cleaves between Arg-Lys of the pro leader sequence. This is rapidly followed by cleavage of Glu-Ala repeats, if present, by the Stel3 protein (Brake, et al, 1984).

When the hybrid precursor polypeptide sequence of the present invention comprises a leader peptide sequence, such as the alpha -factor leader sequence, there can be a processing site immediately adjacent to the 3′ end of the leader peptide sequence. This processing site enables a proteolytic enzyme native to the yeast host cell to cleave the yeast secretion leader peptide sequence from the 5′ end of the native N-terminal propeptide sequence of the mature heterologous protein of interest, when present, or from the 5′ end of the peptide sequence for the mature heterologous IFNαD. The processing site can comprise any peptide sequence that is recognized in vivo by a yeast proteolytic enzyme such that the mature heterologous protein of interest can be processed correctly. The peptide sequence for this processing site may be a naturally occurring peptide sequence for the native processing site of the leader peptide sequence. More preferably, the naturally occurring processing site will be modified, or the processing site will be synthetically derived, so as to be a preferred processing site as described above.

A preferred α-factor prepro signal sequence is shown in SEQ ID NO:5, corresponding to the S. cerevisiae α-factor signal sequence.

b. Alternative Signal Sequences

Suitable signal sequences may also include P. pastoris Acid Phosphatase (PHOI) and PHA-E from the plant lectin Phaseolus vulgaris agglutinin (Cereghino and Cregg, 2000; and Raemaekers, et al., 1999). In addition, the signal sequence may be synthetically derived, or determined from genomic or cDNA libraries using hybridization probe techniques available in the art (see Sambrook et al. (1989).

In accordance with the invention as stated above, the yeast signal peptide and secretion leader peptide sequences represent those parts of the hybrid precursor polypeptide of the invention that can direct the sequence for the mature heterologous IFNαD through the secretory pathway of a yeast host cell.

iii. Human IFNαD peptide coding sequence

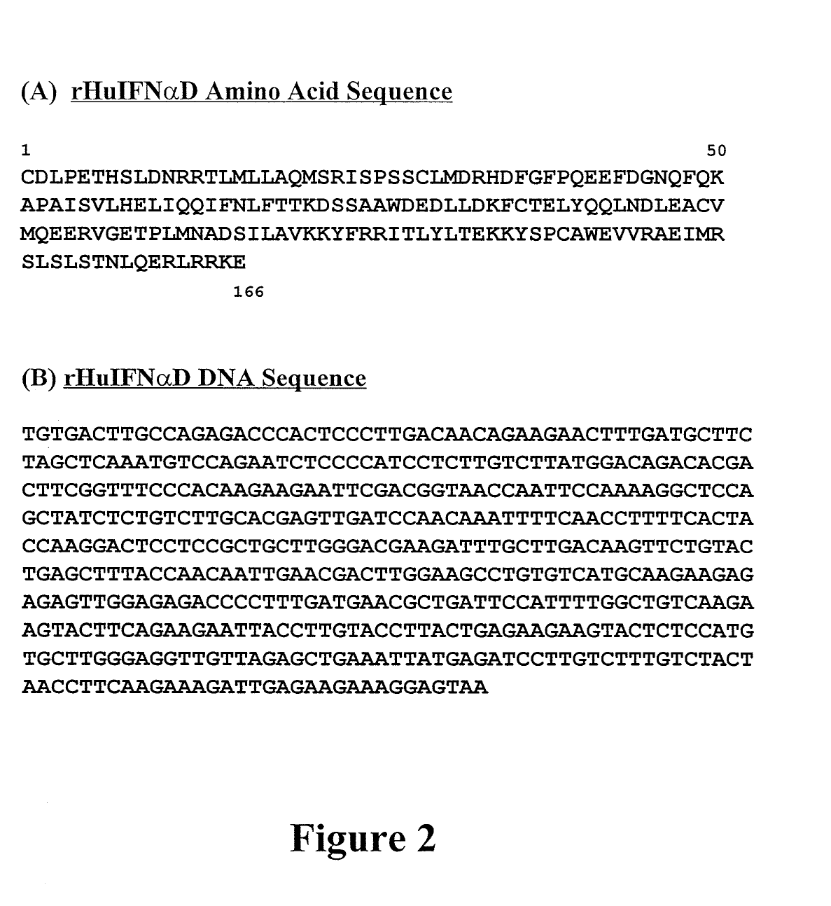

FIG. 2 shows the DNA coding sequence, identified herein as SEQ ID NO: 1, and the corresponding 166 amino acid sequence, identified herein as SEQ ID NO:2, for human IFNαD. The origin of the coding sequences is discussed in Example 2.

Where appropriate, the nucleotide sequence encoding the human IFNαD peptide and any additional nucleotide sequences of interest may be optimized for increased expression in the transformed yeast. That is, these nucleotide sequences can be synthesized using yeast-preferred codons for improved expression. Methods are available in the art for synthesizing yeast-preferred nucleotide sequences of interest (see, for example, U.S. Pat. Nos. 5,219,759 and 5,602,034).

Additional sequence modifications are known to enhance expression of nucleotide coding sequences in yeast hosts. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the nucleotide coding sequence is modified to avoid predicted hairpin secondary mRNA structures.

iv. Transcription and Translation Terminators

The termination regulatory region of the expression cassette may be native with the transcription initiation region, or may be derived from another source, providing that it is recognized by the yeast host. The termination regions may be those of the native alpha -factor transcription termination sequence, or another yeast-recognized termination sequence, such as those for the enzymes mentioned above. The transcriptional termination region may be selected, particularly for stability of the mRNA, to enhance expression.

Preferably the transcription terminator is the Mat- alpha (alpha -factor) transcription terminator. More preferably the transcription termination sequence is the AOX1 transcription termination region as shown in FIG. 1.

v. Selectable Markers

Selectable markers include the biosynthetic pathway genes HIS4 from either P. pastoris or S. cerevisiae, ARG4 from S. cerevisiae, and the Sh ble gene from Streptoalloteichus hindustanus which confers resitance to the bleomycin-related drug Zeocin (Cregg et al., 1985; Cregg and Madden, 1989; and Higgins, et al., 1998). A more recently developed set of biosynthetic markers includes the P. pastoris ADE1 (PR-amidoimidazolesuccinocarboxamide synthase), ARG4 (argininosuccinate lyase), and URA3 (orotidine 5′-phosphate decarboxylase) genes.

The pPEPhEFNαD vector illustrated in FIG. 1 contains the Sh ble gene, thus conferring Zeocin resistance to the P. pastoris host into which it is transformed, as described in Example 3.

vi. Construction of Transforming Vector

As is the case for the heterologous protein IFNαD, each of the other elements present in the hybrid precursor polypeptide can be a known naturally occurring polypeptide sequence or can be synthetically derived, including any variants thereof that do not adversely affect the function of the element as described herein. By “adversely affect” is intended that inclusion of the variant form of the element results in decreased bioactivity of the secreted mature heterologous protein of interest relative to the hybrid precursor polypeptide comprising the native form of the element.

In preparing the expression cassette, the various nucleotide sequence fragments may be manipulated, so as to provide for the sequences in the proper orientation and, as appropriate, in the proper reading frame. Toward this end, adapters or linkers may be employed to join the nucleotide fragments or other manipulations may be involved to provide for convenient restriction sites, removal of superfluous nucleotides, removal of restriction sites, or the like. For this purpose, in vitro mutagenesis, primer repair, restriction, annealing, resubstitutions, e.g., transitions and transversions, may be involved. See particularly Sambrook et al. (1989).

The expression cassettes of the present invention can be ligated into a replicon (e.g., plasmid, cosmid, virus, mini-chromosome), thus forming an expression vector that is capable of autonomous DNA replication in vivo. Preferably the replicon will be a plasmid. Such a plasmid expression vector will be maintained in one or more replication systems, preferably two replications systems, that allow for stable maintenance within a prokaryotic host for cloning purposes and integration within a yeast host cell for expression purposes.

Additionally, a plasmid expression vector may be integrated as a high or low copy number plasmid. A strain that contains multiple integrated copies of an expression cassette can sometimes yield more heterologous protein than single copy strains (Clare, et al., 1991).

FIGS. 2 shows an exemplary tranformation vector for use in transforming yeast cells capable of expressing mature, active IFNαD in yeast cell culture. Details of the vector construction are given in Example 2.

B. Transformation of Yeast Cells

Yeast cells are transformed with expression constructs described above using a variety of standard techniques including, but not limited to, electroporation, microparticle bombardment, spheroplast generation methods, or whole cell methods such as those involving lithium chloride and polyethylene glycol (Cregg et al., 1985; Liu et al., 1992; Waterham et al., 1996; and Cregg and Russell, 1998).

Example 3 describes an exemplary method of transforming P. pastoris with the pPEPhIFNαD plasmid illustrated in FIG. 1.

C. Culturing and Obtaining Secreted human IFNαD

Transformants are grown in an appropriate nutrient medium, and, where appropriate, maintained under selective pressure to insure retention of endogenous DNA. Where expression is inducible, growth can be permitted of the yeast host to yield a high density of cells, and then expression is induced. Example 4 describes an exemplary method of inducing human IFNαD expression in P. pastoris.

In accordance with an important aspect of this invention, it has been found that the transformed P. pastoris is able to express and secrete human IFNαD having a specific activity of 1.7×10⁸ U/mg protein as disclosed in Example 7. In one embodiment, the specific activity of the protein is at least about 1.0×10⁷ U/mg protein. In another embodiment, the specific activity of the human IFNαD is at least about 1.0×10⁸ U/mg. In yet another embodiment, the specific activity is at least about 1.5×10⁸ U/mg. In yet, still another embodiment, the specific activity is at least about 2-5×10⁸ U/mg. The specific activity may be at least about 1×10⁹ U/mg. The determination of specific activity can be performed by antiviral assays which are known to those 5 skilled in the art. An exemplary antiviral assay is discribed in Pontzer and Johnson, 1985, which is hereby incorporated by reference in its entirety.

The small-scale expression experiments described in Example 4 used 10 ml shaker cultures with an initial OD₆₀₀ of 2. These experiments produced a total of 3 mg to 7 mg of biologically active rHuIFNαD per 10 ml of culture supernatant. However, the present invention also contemplates the ability of P. pastoris to grow to high densities in a fermenter. This creates an even greater potential to produce large amounts of relatively pure and functionally active rHuIFNαD that can easily be recovered from the culture supernatant fractions.

D. Isolating Secreted human IFNαD

One of the major advantages of expressing rHuIFNαD as a secreted protein in Pichia pastoris is that Pichia secretes very low levels of contaminating native proteins in the culture medium. That, combined with the use of the minimal Pichia growth medium with low protein content, led to the efficient secretion of a biologically active rHuIFNαD that constitutes the vast majority of the total protein in the medium.

In essence, the very nature of this secreted expression system allows for simply the secretion of the protein into the medium to serve as the first step in the purification process. This results in a straightforward and simple procedure for further purification using techniques such as molecular sieve and ion-exchange chromatography, electrophoresis, dialysis, solvent-solvent extraction, and the like. Such methods are known in the art and described for example in Deutscher, 1990 and Scopes, 1982.

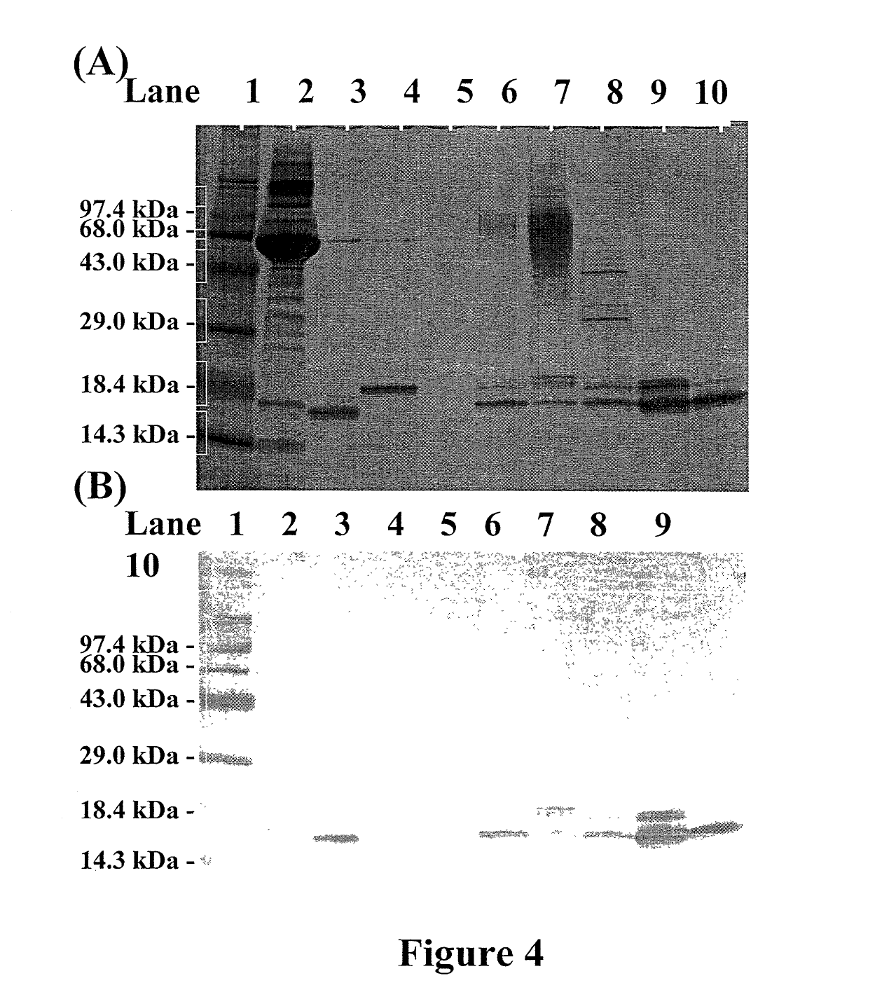

As described in Example 6, one round of molecular sieve chromatography essentially eliminated the higher molecular weight contaminating proteins leaving behind a rHuIFNαD that is ≧99% pure with a very high antiviral activity. FIG. 4B illustrates that two rHuIFNαD minor bands migrated slightly higher than the major band. N-terminal sequencing has confirmed that these bands are rHuIFNαD molecules with a N-terminal 9- and 11 -amino acid extensions from the α-factor signal sequence. It is worthy to note, however, that the presence of these two additional bands did not seem to negatively affect the biological activity of the rHuIFNαD preparations.

TABLE 2

Sequences Provided In Support Of The Invention.

| | |
|---|---|
| Description | SEQ. ID NO. |

| | |
|---|---|
| Human IFNαD DNA Sequence—FIG. 2 | 1 |
| Human IFNαD Amino Acid Sequence—FIG. 2 | 2 |
| 5′ AOX1 Primer Sequence | 3 |
| 5′-GACTGGTTCCAATTGACAAGC-3′ | |
| 3′ AOX1 Primer Sequence | 4 |
| 5′-GCAAATGGCATTCTGACATCC-3′ | |
| α-factor Signal Sequence | 5 |
| AKEEGVSLEKR | |
| IFNα-N7 Amino Acid Sequence—SEQ ID NO:17 in U.S. Pat. No.: 6,204,022 which | 6 |
| is incorporated herein by reference. | |
| Primer PM-Gd1 | 7 |
| 5′-GAA GAA CTT TGA TGC TTC TAG CTA GAA TGA ACA GAT TGT CCC C-3′ | |
| Primer PM-Gd2 | 8 |
| 5′-GGG GAC AAT CTG TTC ATT CTA GCT AGA AGC ATC AAA GTT CTT C-3′ | |
| Primer NLVg(DR).MUT1 | 9 |
| 5′-GAT GCT TCT AGC TCA AAT GAA CAG ATT GTC CCC ACA CTC TTG TC-3′ | |
| Primer NLVg(DR).MUT2 | 10 |
| 5′-GAC AAG AGT GTG GGG ACA ATC TGT TCA TTT GAG CTA GAA GCA TC-3′ | |
| Primer NLVgDRN.MUT1 | 11 |
| 5′-GAA CTT TGA TGC TTC TAG CTC AAA TGT CCA GAT TGT CCC CAC AC-3′ | |
| Primer NLVgDRN.MUT2 | 12 |
| 5′-GTG TGG GGA CAA TCT GGA CAT TTG AGC TAG AAG CAT CAA AGT TC-3′ | |
| Primer NLVgNLH.MUT1 | 13 |
| 5′-GCT CAA ATG TCC AGA ATC TCC CCA TCC TCT TGT CTT ATG GAC AG-3′ | |
| Primer NLVgNLH.MUT2 | 14 |
| 5′-CTG TCC ATA AGA CAA GAG GAT GGG GAG ATT CTG GAC ATT TGA GC-3′ | |

### V. EXAMPLES

The following examples further illustrate the invention described herein and are in no way intended to limit the scope of the invention.

### Example 1

A. Materials:

SacI restriction endonuclease was purchased from Boehringer Mannheim (Indianapolis, Ind.). All P. pastoris plasmid and yeast expression reagents were purchased from Invitrogen (San Diego, Calif.). All oligonucleotide primers, including primers for mutagenesis and the 5′AOX1 and 3′AOX1 primers for colony PCR screening and sequencing, were synthesized by Life Technologies (Gaithersburg, Md.) (5′AOX1 primer sequence: 5′-GACTGGTTCCAATTGACAAGC-3′ (SEQ ID NO:3); 3′AOX1 primer sequence: 5′-GCAAATGGCATTCTGACATCC-3′ (SEQ ID NO:4)). All PCR reagents excluding the primers were purchased from Perkin-Elmer/Roche (Branchburg, N.J.). Microcon-100, Centricon-20 and Micropure-EZ filter units were from Millipore Corporation (Bedford, Mass.). ZymoLyase was from Seikagaku, America (Ijamsville, Md.). rHuIFNαA and rHuIFNαD standards were purchased from PBL (New Brunswick, N.J.) and Biosource International (Camarillo, Calif.), respectively. DNA sequencing was performed by the dideoxy chain-termination method using ABI 373 DNA sequencer and ABI PRISM BigDye Terminator Cycle Seqeuncing Kit (PE Biosystems Inc., Foster City, Calif.). The N-terminal sequence of the protein was determined by using Edman degradation on an ABI 494 Procise Sequenator (PE Biosystems Inc., Foster City, Calif.). DNA sequencing and N-terminal protein sequencing were both performed at the PAN Facility at Stanford University. Yeast genomic DNA was isolated using the method outlined in the 5 Prime, 3 Prime PerfectYeast gDNA Isolation kit (5 Prime, 3 Prime, Inc., Boulder, Colo.).

B. Strains and Plasmids:

INVαF′ One Shot chemically competent E. Coli strain (Genotype: F′ endA1 recA1 hsdR17 (rₖ⁻, mₖ⁺) supE44 thi-1 gyrA96 relA1 φ80lacZΔM15Δ(lacZY A-argF)U169γ), plasmid pPICZα and Pichia pastoris X-33 wildtype yeast strain were purchased from Invitrogen (San Diego, Calif.). Plasmid pPICZα(IFNα-N7) contains a modified HuIFN-αD synthetic gene inserted into the XhoI and NotI sites of the pPICZα. Preparation of pPICZα(IFNα-N7) is described in U.S. patent application No. 08/954,395, filed Oct. 20, 1997, which is expressly incorporated by reference in its entirety herein.

### Example 2

Construction of a Transforming Vector Containing the HuIFNαD Gene Sequence

A. Vector Construction:

In previous experiments, a series of synthetic hybrid interferon genes were constructed using combinations of HuIFNαb 1 and ovine IFNτ(oIFNτ) sequences for structure-function analyses (U.S. Pat. No: 6,204,022 (Johnson et al.), issued Mar. 20, 2001, which is expressly incorporated by reference in its entirety herein). The hybrid genes were designed to include Pichia preferred codon usage and were inserted into the pPIZα expression vector (Invitrogen, San Diego, Calif.) using the Xho I and Not I restriction enzyme sites.

The pPICZα vector features the AOX1 gene promoter to drive the expression of the heterologous protein, the a mating factor prepro signal sequence to target the protein to the secretory pathway, and the Zeocin™ resistance gene for positive selection of the recombinant clones in E. coli and P. pastoris. One of the hybrid genes products, IFNα-N7, corresponding to SEQ ID NO:6, differed from HuIFNα1 amino acid sequence by only five amino acids. The IFNα-N7 hybrid gene is described in U.S. Pat. No. 6,204,022, which was incorporated by reference above. Four consecutive rounds of in vitro site-directed mutagenesis were performed as described below in order to back-mutate the hybrid interferon gene in plasmid pPICZα(IFNα-N7) to HuIFNαD. The resulting plasmid, called pPEPhIFNαD, includes all the elements of the pPICZα vector as well as the HuIFNαD gene sequence (FIG. 1). The DNA sequence of the gene insert in the pPEPhIFNα was confirmed by DNA sequence analysis (FIG. 2).

B. In Vitro Site-Directed Mutagenesis:

Site-directed mutagenesis was performed using a PCR-based in vitro site-directed mutagenesis method outlined in the QuickChange™ Site-Directed Mutagenesis Kit (Strategene, San Diego, Calif.). Four primer pairs were synthesized and used in four consecutive mutagenesis reactions to change plasmid pPICZα(IFNα-N7) to pPEPhIFNαD. Each of the primers used in the first primer pair used in round 1 was 43 bases in length (primer PM-Gd1 and primer PM-Gd2, corresponding to SEQ ID NOS:7 and 8, respectively), while the rest of the primer pairs used were 44 bases (round 2: NLVg(DR).MUT1 and NLVg(DR).MUT2 [SEQ ID NOS:9 and 10, respectively]; round 3: NLVgDRN.MUT1 and NLVgDRN.MUT2 [SEQ ID NOS: 11 and 12, respectively]; and round 4: NLVgNLH.NMtT1 and NLVgNLH.MUT2 [SEQ ID NOS:13 and 14, respectively].

Clones containing plasmids with the desired mutation for each mutagenesis reaction were identified by bacterial colony PCR screening using the 5′ and 3′ AOX1 primers, followed by DNA sequence analysis of the resulting PCR products. The colony PCR reaction mixture was done in a total volume of 50 μl consisting of 20 μl of resuspended bacterial colony, 10 pmoles each of the 5′ and 3′ AOX1 primers, 2.5 mM MgCl₂, 125 μM of dATP, 125 μM of dTTP, 125 μM of dCTP, 125 μM of dGTP, 1X PCR Buffer II (50 mM KCl, 10 mM Tris-HCl, pH 8.3). PCR amplification was done for 30 cycles (each for 1 minute at 95° C., 1 minute at 54° C. and 1 minute at 72° C.) with a final extension of 7 minutes at 72° C. on a Perkin Elmer DNA Thermal Cycler 480.

### Example 3

Transformation of pPEPhIFNαD into P. pastoris

A. Preparation of Mut⁺ pPEPhI-FNαD Strains

Plasmid pPEPhIFNαD was linearized with Sac I restriction endonuclease prior to electroporation into the X-33 strain of Pichia pastoris. A Sac I linearized pPICZα vector with no gene insert was also used as a control. In this system, homologous recombination can occur within the upstream 5′ sequence of the AOX1 promoter region or downstream AOX1 transcription termination region (TT) (FIG. 1). Depending on whether the recombination event was a single crossover event (insertion) or a double crossover event (replacement), the positive transformants will contain either an intact or deleted endogenous AOX1 gene, respectively.

Positive transformants in which the recombination event resulted in the deletion of the endogenous AOX1 gene have a slow growth phenotype on methanol medium and have been referred to as “methanol utilization slow” (Mut^{S}) colonies. Positive transformants with the intact AOX1 gene have been designated as “methanol utilization plus” (Mut⁺) colonies. Mut⁺ and Mut^{S} colonies can be easily distinguished from one another by the presence or absence of specific PCR products derived from colony PCR of the Zeocin™ resistant transformants using the 5′AOX1 and 3′AOX1 PCR primers.

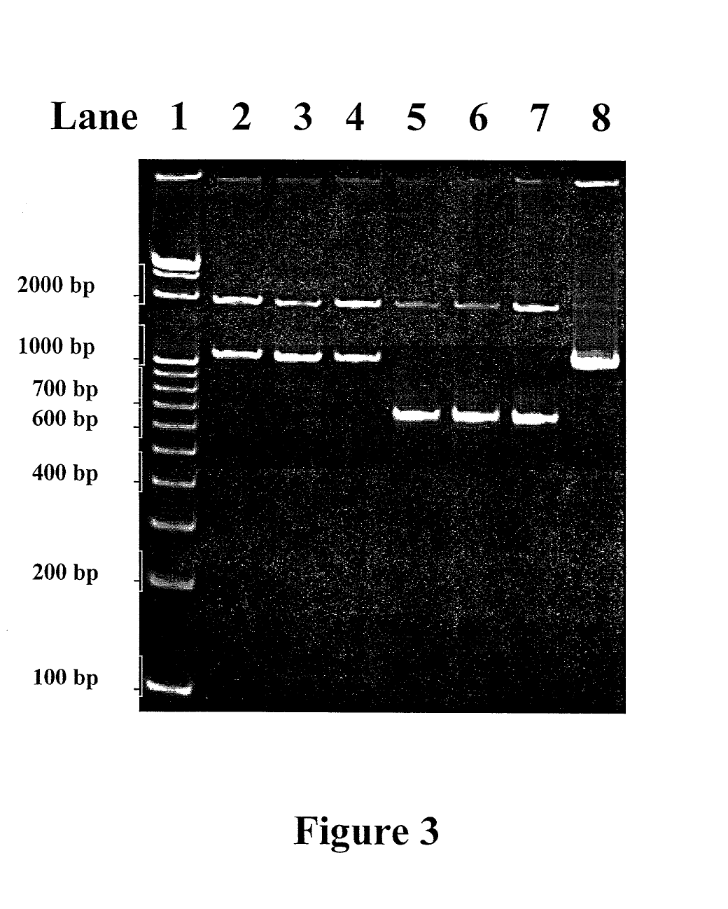

Colony PCR of Mut⁺ colonies resulted in the visualization of a 1029 base pair (b.p.) band and an approximately 2200 b.p. band (FIG. 3). These bands represent genomic amplification products that include the HuIFNαD gene insert or the intact endogenous copy of the AOX1 gene, respectively. The absence of the 2200 b.p. PCR product indicates a Mut^{S} colony. The presence of an approximately 650 b.p. band on a 6% PAGE rather than the 1029 b.p. band in the pPICZα vector control transformed colonies confirmed the absence of the gene insert which is 501 b.p. long (FIG. 3). Note that the approximately 650 b.p. PCR product derived from the pPICZα transformants is actually 593 b.p. and migrates as such on an agarose gel (data not shown). This anomalous migration pattern may be due in part to the variance in resolving power between polyacrylamide and agarose gels (Sambrook, 1989). In these experiments, 93% (13 of 14) of the pPEPhIFNuαD transformants and 100% (2 of 2) of the control pPICZα transformants that were screened were Mut⁺. Eight of the Mut⁺ pPEPhIFNαD colonies and one of the Mut⁺ control pPICZα colonies were subsequently used for small-scale expression studies.

B. Pichia pastoris Transformation and Expression Experiments:

The procedure for the transformation and expression experiments are as outlined in the Invitrogen EasySelect manual. Selected positive transformants were grown overnight in BMGY medium, then the cells were induced using an OD₆₀₀ of 2 in BMMY medium. The cultures were fed with 1 ml of 10% methanol at 24 and 48 hours post-induction. After 72 hours of culture, the entire culture supernatant was harvested, filter-sterilized and analyzed by one-dimensional SDS-polyacrylamide gel electrophoresis using a 14% Tris-Glycine gel.

Proteins were visualized by Colloidal Coomassie staining (Novex, San Diego, Calif.). A duplicate gel was transblotted onto a PVDF membrane (Millipore Corporation, Bedford, Mass.) for Western blotting. The rHuIFNαD protein was detected on the blot by incubation with an anti-human IFNα mouse monoclonal antibody (clone MMHA-3), followed by a biotinylated goat anti-mouse antibody (Biosource International, Camarillo, Calif.) and a colorimetric detection system using streptavidin-alkaline phosphatase enzyme conjugate, nitroblue tetrazolium (NBT) and 5′-bromo-4-chloro-3-indolylphosphate (BCIP).

### Example 4

HuIFNαD Induction

The selected Mut⁺ colonies were induced for expression by growth in BMMY, a methanol-containing medium. The supernatants were harvested after three days of induction. FIG. 4A shows a Coomassie stained SDS-PAGE gel with protein samples that are representative of recombinant pPICZα control and HuIFNαD day 3 supernatants (lanes 5 and 6, respectively). The HuIFNαD supernatant shows the presence of a protein band that migrates slightly above the rHuIFNαA standard and below the roIFNτ standard. This band is absent in the pPICZα control supernatant.

This result corresponds to the amino acid lengths and theoretical molecular weights of these proteins. The amino acid lengths and theoretical molecular weights for rHuIFNαA, roIFNτ and rHuIFNαD are 165 amino acids and 19.2 kDa, 172 amino acids and 19.9 kDa, and 166 amino acids and 19.4 kDa, respectively. These theoretical protein molecular weight values were slightly higher than the estimations based upon the prestained molecular weight markers (FIG. 4A and 4B, Lane 1). In our hands, the prestained molecular weight markers have often demonstrated faster migration rates than predicted. The dyes linked to the molecular weight standards most likely account for this discrepancy.

### Example 5

Western Blot Analysis of HuIFNαD

Using an anti-human IFNα specific monoclonal antibody, western blot analysis after electrotransfer of a duplicate gel from FIG. 4A confirmed that the putative rHuIFNαD protein band was indeed an interferon molecule and has the same electrophoretic mobility as the rHuIFNαD standard reference molecule (FIG. 4B, lanes 6 and 2, respectively). Two additional bands migrating at slightly slower rates than the major band also reacted with the anti-human IFNα antibody. N-terminal sequencing revealed that these bands represent rHuIFNαD with N-terminal α-factor signal extensions. FIG. 4B also shows that immunoblotting did not detect any degradation products of the secreted rHuIFNαD.

This indicates that rHuIFNαD is resistant to P. pastoris proteinases during expression. Note that the presence of additional nonimmunoreactive bands in the rHuIFNαD standard lane (FIG. 4A, lane 2) are most likely BSA protein bands, since the rHuIFNαD standard is in a buffered solution with 0.1% BSA (FIG. 4B, lane 2). All eight rHuIFNαD colonies tested secreted one major band and two minor bands that reacted with the HuIFNα antibody (data not shown).

### Example 6

Purification and Characterization of HuIFNαD

A. Purification of rHuIFNαD from Pichia Supernatants:

Culture supernatants (20 ml volume) were buffer exchanged with 1 mM Tris, 150 nM NaCl, and concentrated to a final volume of 2 ml with a Centricon Plus-20 (Millipore Corporation, Bedford, Mass.) prior to loading onto a HiPrep Sephacryl 26/60 S-100 High Resolution size exclusion column at 4° C. (Pharmacia, Peapack, N.J.). The first 120 ml flowthrough following the injection of the sample were collected at 1 ml/minute and designated fraction 1. Three fractions of 20 mls each were collected and designated fraction 2, 3, and 4 respectively. The fractions were concentrated to 1 ml and were run on 14% SDS-PAGE gels and stained using the Novex Colloidal Blue Staining Kit. The Alphalmager 2000 software (Alpha Innotech, San Leandro, Calif.) was used to determine the band density ratios for densitometric analysis and estimations of isoelectric points (pI) from isoelectric focusing gels. The Lasergene software (DNAStar, Madison, Wis.) was used to do all DNA analysis. Sample total protein concentration was determined using the bicinchoninic acid (BCA) assay kit (Pierce, Rockford, Ill.).

The Coomassie stained SDS-PAGE gel, shown on FIG. 4A, also includes purified fractions of a rHuIFNαD day 3 supernatant from the best producing transformant, following one round of molecular sieve chromatography (lanes 7 to 10). Most of the high molecular weight proteins in fraction 3 and fraction 4 were no longer detectable by Coomassie staining (FIG. 4A) nor silver staining (data not shown) of SDS-PAGE gels (N=4). As observed with the unpurified supernatants, western blot analysis of purified fraction 3 verified three immunoreactive proteins—one major band and two other bands with slightly higher migration patterns (FIG. 4B, lane 9).

Note that although fraction 4 showed only 2 bands that were detected by Coomassie staining and immunoblotting (FIG. 4A and 4B, lane 10), the third band was clearly evident in silver stained SDS-PAGE gels (data not shown). Densitometric analysis demonstrated that the combined band density ratios of the three HuIFNα specific bands amounted to ≧99% of the total protein in the sample fraction 3 and sample fraction 4. The band density ratio of just the major band in fraction 3 and fraction 4 was about 62% and 89%, respectively.

B. N-Terminal Sequencing and IEF

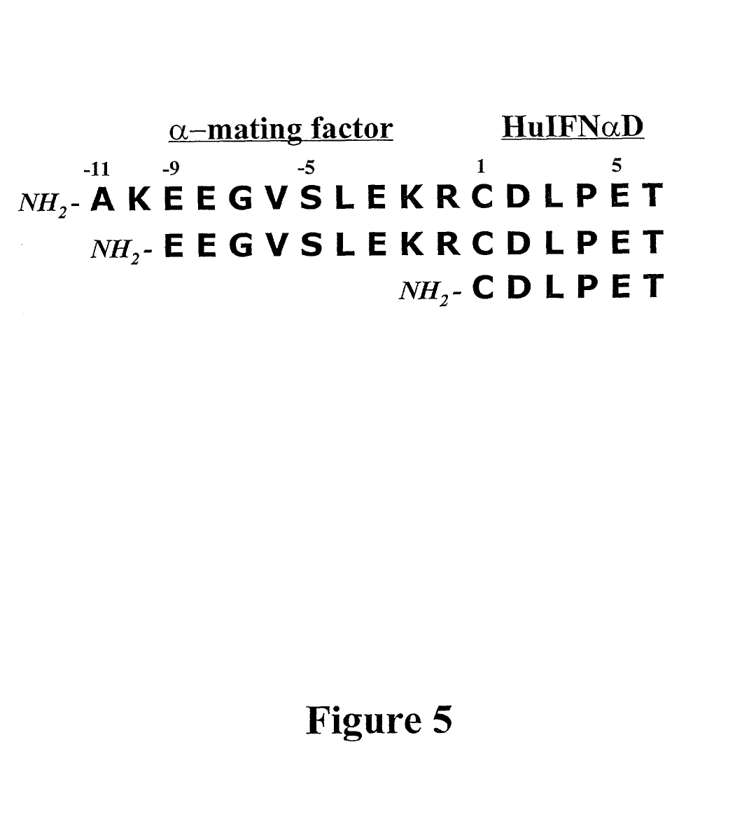

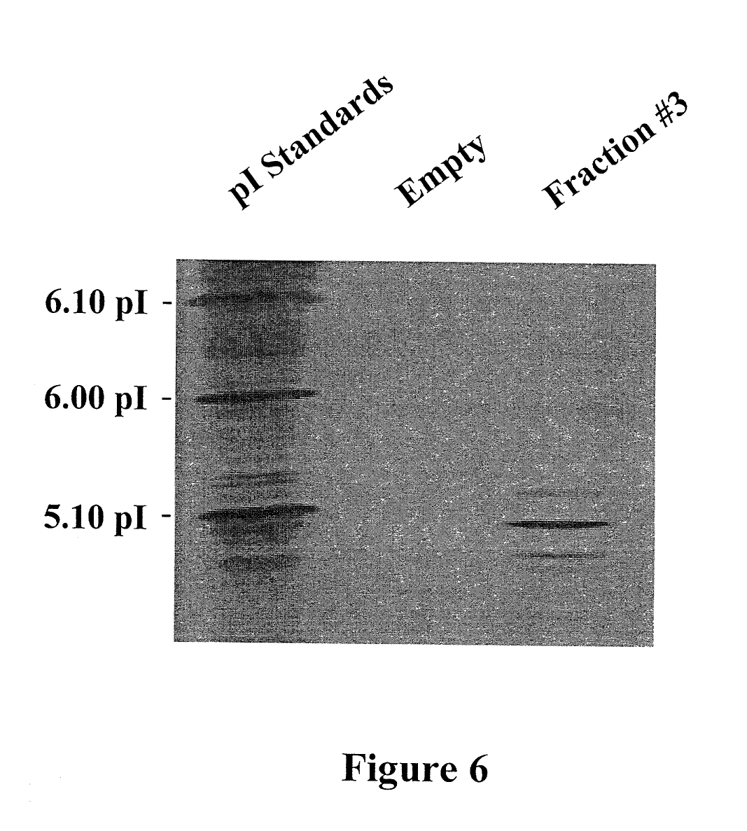

N-terminal microsequencing analysis of the two higher molecular weight bands revealed an additional 9 amino acid or 11 amino acid N-terminal extension from the alpha mating factor signal sequence, whereas, the major band revealed correct processing of the signal sequence and showed an exact match at amino acid positions 2 through 20 with that of the native HuIFNαD sequence (FIG. 5). As expected, the highly reactive cysteine at the N-terminus could not be confirmed because the sample was not reduced nor alkylated prior to N-terminal sequencing. The cysteine at amino acid position 1 was indirectly verified by DNA sequencing of the HuIFNαD gene integrated in the genome of the recombinant HuLFNαD P. pastoris clones used for the expression studies. The isoelectric focusing (IEF) gel shown on FIG. 6 depicts the relative band mobilities, which represents isoelectric points (pI), of the three bands present in fraction 3. The theoretical pI of rHuIFNαD is 5.077. Computer generated calculations of the pI of the major band in purified fraction 3, based upon the band mobility on the IEF gels, was estimated to be about 5.06.

Isoelectric focusing (IEF) was performed using 1.0 mm thick pre-cast IEF vertical gels (5% acrylamide with 2% ampholytes, pH 3-7) (Novex, San Diego, Calif.). The upper chamber cathode buffer used was 40 mM lysine and the lower chamber anode buffer was 7 mM Phosphoric Acid. The gel was run for one hour at a 100V constant, then 1 hour for 200V, and followed by 500V for 30 minutes. Following the run, the gel was fixed using 12% TCA, 3.5% sulfosalicylic acid for 1 hour then stained with Colloidal Coomassie stain.

### Example 7

Antiviral Activity of HuIFNαD

The amount of biologically active rHuIFNαD secreted by P. pastoris was determined by measuring the antiviral activity in the purified fractions using a standard cytopathic protection assay. Vesicular stomatitis virus challenge of Madin Darby bovine kidney (MDBK) cells was performed to quantitate the antiviral activity as described in Pontzer and Johnson (1985). Typically, the total protein concentration in these purified fractions ranged from approximately 0.3 mg/ml to 0.7 mg/ml. Measuring the antiviral activity of the rHuIFNαD in the same fractions demonstrated that the rHuIFNαD purified from P. pastoris supernatants had full biological activity with specific activity of about 1.7×10⁸ U/mg. This is greater than two times higher than the antiviral activity determined for the commercially available rHuIFNαD with specific activities ranging from 5.0-7.5×10⁷ antiviral units/ mg. Clearly, these results further substantiate the finding that the rHuIFNαD secreted by P. pastoris is superior structurally as well as functionally when compared to the commercially available and previously disclosed rHuIFNαD.

Although the invention has been described with respect to particular embodiments, it will be apparent to those skilled in the art that various changes and modifications can be made without departing from the invention.

It is claimed:

1. A method for producing human IFNαD comprising
(a) transforming Pichia pastoris with a vector comprising a nucleotide sequence that comprises in the 5′ to 3′ direction and operably linked
(i) a P. pastoris-recognized transcription and translation initiation region,
(ii) a signal peptide sequence for a P. pastoris secreted protein,
(iii) a peptide coding sequence for said human IFNαD, or variant thereof, and
(iv) a P pastoris-recognized transcription and translation termination region;
(b) culturing said transformed P. pastoris cells,
(c) by said culturing, obtaining IFNUαD protein in secreted form in the extracellular culture medium at a specific activity of at least 1.7×10⁸ U/mg protein, and
(d) isolating the IFNαD protein from the extracellular culture medium.

2. The method of claim 1, wherein said transcription and translation initiation region is the Pichia pastoris AOX1 promoter.

3. The method of claim 1, wherein said transcription and translation initiation region is the promoter selected from the group consisting of Pichia pastoris GAP, FLD1, PEX8, and YPT1 promoters.

4. The method of claim 1, wherein said signal peptide sequence is a signal peptide sequence for a Saccharomyces cerevisiae α-factor.

5. The method of claim 1, wherein said variant has an amino acid sequence that has at least about 95% sequence identity to the amino acid sequence of human-IFNαD.

6. The method of claim 1, wherein said variant has an amino acid sequence that has at least about 98% sequence identity to the amino acid sequence of human-IFNαD.

7. The method of claim 1, wherein said variant has an amino acid sequence that has at least about 99% sequence identity to the amino acid sequence of human-lFNαD.

8. The method of claim I, wherein said transcription and translation termination region is the AOX1 termination region.

9. An isolated human IFNAαD protein prepared by
(a) transforming Pichia pastoris with a vector comprising a nucleotide sequence that comprises in the 5′ to 3′ direction and operably linked
(i) a P. pastoris-recognized transcription and translation initiation region,
(ii) a signal peptide sequence for a P. pastoris secreted protein,
(iii) a peptide coding sequence for said human IFNαD, or variant thereof, and
(iv) a P pastoris-recognized transcription and translation termination region;
(b) culturing said transformed P. pastoris cells,
(c) by said culturing, obtaining IFNαD protein in secreted form in the extracellular culture medium at a specific activity of at least 1.7×10⁸ U/mg protein, and
(d) isolating the IFNαD protein from the extracellular culture medium.

10. The human IFNαD protein of claim 9, wherein said transcription and translation initiation region is the Pichia pastoris AOX1 promoter.

11. The human IFNαD protein of claim 9, wherein said transcription and translation initiation region is the promoter selected from the group consisting of Pichia pastoris GAP, FLD1, PEX8, and YPT1 promoters.

12. The human IFNαD protein of claim 9, wherein said signal peptide sequence is a signal peptide sequence for a Saccharomyces cerevisiae α-factor.

13. The human IFNαD protein of claim 9, wherein said variant has an amino acid sequence that has at least about 95% sequence identity to the amino acid sequence of human-IFNαD.

14. The human IFNαD protein of claim 9, wherein said variant has an amino acid sequence that has at least about 98% sequence identity to the amino acid sequence of human-IFNαD.

15. The human IFNαD protein of claim 9, wherein said variant has an amino acid sequence that has at least about 99% sequence identity to the amino acid sequence of human-IFNαD.

16. The human IFNαD protein of claim 9, wherein said transcription and translation termination region is the AOX1 termination region.
